(19) United States
(12) Patent Application Publication
(10) Pub. No.: US 2007/0010550 A1
(43) Pub. Date: Jan. 11, 2007

(54) Scopolamine to Reduce or Eliminate Hot Flashes, Night Sweats, and Insomnia

(75) Inventor: Shirlyn B. McKenzie, San Antonio, TX (US)

Correspondence Address: MONICA A. DE LA PAZ;FULBRIGHT & JAWORSKI L.L.P., 600 CONGRESS AVENUE, SUITE 2400, AUSTIN, TX, 78701, US

(73) Assignee: THE BOARD OF REGENTS OF THE UNIVERSITY OF TEXAS

(21) Appl. No.: 11/421,960

(22) Filed: Jun. 2, 2006

Related U.S. Application Data
(60) Provisional application No. 60/697,718, filed on Jul. 8, 2005.

(51) Int. Cl. A61K 31/46

(52) U.S. Cl. 514/304

(57) ABSTRACT

Compositions and methods of treating or preventing hot flashes, night sweats, and/or insomnia in a subject that involve scopolamine are disclosed. For example, methods of treating hot flashes, night sweats, and/or insomnia in a subject that involve transdermal delivery of scopolamine are set forth herein.

This application claims the benefit of the filing date of U.S. provisional patent application Ser. No. 60/697,718, filed Jul. 8, 2005, the entire contents of which is hereby specifically incorporated by reference in its entirety.

### BACKGROUND OF THE INVENTION

1. Field of the Invention

The present invention relates generally to the fields of pharmacology, pharmaceutics, and clinical medicine. More particularly, it concerns methods of treating or preventing hot flashes, night sweats, and/or insomnia in a subject that involves administering to the subject a therapeutically effective amount of scopolamine. It also concerns compositions comprising scopolamine that can be applied in the treatment of hot flashes, night sweats, and/or insomnia in a subject. For example, the scopolamine can be formulated in a transdermal delivery device.

2. Description of Related Art

Hot flashes are experienced by many individuals. A hot flash is a feeling of warmth, sometimes associated with flushing, that spreads over the body and may be accompanied by perspiration.

Perhaps the most common cause of hot flashes is the hormonal changes associated with menopause. Hot flashes may last for a decade or more in some women. There is no way to predict when hot flashes will cease in an individual woman. Although they often decrease in frequency with time, they may persist for many years in some women. Although the cause of hot flashes is not completely understood, they may be related more to the fluctuations of hormone levels as opposed to low hormone levels per se.

In menopause, hot flashes are triggered by natural hormonal fluctuations that occur as the ovaries stop producing the female hormone estrogen. For pre-menopausal breast cancer survivors, hot flashes may be the result of early artificial menopause triggered by chemotherapy and/or radiation therapy. In addition, many women who have been treated for breast cancer also take the estrogen-like drug tamoxifen for five years after completion of their initial treatment in an effort to prevent cancer recurrence. Tamoxifen use has been associated with hot flashes (reviewed in Mourits et al., 2001). More recently, aromatase inhibitors such as letrozole, are being used in place of tamoxifen to prevent breast cancer recurrence. Aromatase inhibitors have a higher incidence of associated hot flashes than tamoxifen.

Another cause of hot flashes is chemotherapy and/or radiation therapy for treatment of cancer. Many patients who have been treated for breast cancer have hot flashes (Molina et al., 2005).

Although generally associated with women, hot flashes can also occur in men. Data regarding the pathophysiology and management of hot flashes in men with prostate cancer are scant. The limited data that exist suggest that hot flashes are related to changes in sex hormone levels, causing instability in the hypothalamic thermoregulatory center, analogous to hot flashes that occur in women (Spetz et al., 2003).

Other symptoms that are often associated with hot flashes, particularly in menopausal women, include night sweats, vaginal dryness, sleep disturbances and mood alteration.

For healthy women, menopausal symptoms are often alleviated with hormone replacement therapy, such as with estrogen. However, hormone replacement is not generally recommended for women with a history of breast cancer because of concern that it will increase the risk of recurrence.

Alternatives to hormone replacement, such as plant estrogens, are not well-researched for their safety in any women. The proposed mechanism of action of estrogen replacement on hot flash amelioration is by raising the core body temperature sweating threshold (Freedman et al., 2002). However, many women have relative or absolute contraindications to estrogen replacement. In May 2002, the Women's Health Initiative (WHI)(Chlebowski et al.), a large, randomized, placebo-controlled trial of the risks and benefits of estrogen plus progestin in healthy postmenopausal women, was stopped prematurely at a mean follow-up of 5.2 years because of the detection of a significantly increased breast cancer risk in women receiving hormone replacement therapy (Chlebowski et al., 2003). Tumors among women in the hormone replacement therapy group were slightly larger and more advanced than in the placebo group, with a substantial and statistically significant rise in the percentage of abnormal mammograms at first annual screening. (Chlebowski et al., 2003)

Numerous nonestrogenic, pharmacologic treatment interventions for hot flash management in breast cancer patients have been evaluated. Agents that have been evaluated include androgens, progestational agents, selective serotonin reuptake inhibitors (SSRIs), alpha adrenergic agonists (e.g., methyl dopa, transdermal clonidine), beta-blockers, veralipride (an antidopaminergic agent), and vitamin E. Inferior efficacy and side effects limit the use of many of these agents.

As with women with hormonally sensitive tumors, there are concerns about the effects of hormone use on the outcome of prostate cancer, in addition to other well-described side effects.

Scopolamine is a belladonna alkaloid with well-known pharmacological properties. It is an anticholinergic agent which acts (a) as a competitive inhibitor at postganglionic muscarinic receptor sites of the parasympathetic nervous system, and (b) on smooth muscles that respond to acetylcholine but lack cholinergic innervation. It has been suggested that scopolamine acts in the central nervous system (CNS) by blocking cholinergic transmission from the vestibular nuclei to higher centers in the CNS and from the reticular formation to the vomiting center (McEvoy, 1990; Gilman, et al., 1990). Scopolamine can inhibit the secretion of saliva and sweat, decrease gastrointestinal secretions and motility, cause drowsiness, dilate the pupils, increase heart rate, and depress motor function (Gilman et al., 1990).

Scopolamine has been formulated in a transdermal delivery device for the prevention and treatment of nausea and vomiting associated with motion sickness and recovery from anesthesia and surgery.

A transdermal drug delivery device, which may be of an active or a passive design, is a device which provides an alternative route for administering medication. These devices allow for pharmaceuticals to be delivered across the skin barrier. This approach to drug delivery offers many advantages over traditional methods. As a substitute for the oral route, transdermal drug delivery enables the avoidance of gastrointestinal absorption, with its associated pitfalls of enzymatic and pH associated deactivation. This method also allows for reduced pharmacological dosaging due to the shortened metabolization pathway of the transdermal route versus the gastrointestinal pathway. Transdermal delivery devices also permits constant dosing rather than the peaks and valleys in medication level associated with orally administered medications. Multi-day therapy with a single application, rapid notification of medication in the event of emergency, as well as the capacity to terminate drug effects rapidly via patch removal, are all further advantages of this route.

Although oral administration of scopolamine has been suggested for treatment of climacteric complaints in perimenopausal women (Lagrelius et al., 1981), transdermal administration for treatment has not been described. Oral administration of bellergal, a composition of ergotamine tartrate, belladonna alkaloids, and phenobarbital, has been reported to decrease the severity and frequency of hot flashes in symptomatic women. Transdermal administration of bellergal has not been described. Oral administration is limited by peaks and troughs of efficacy, the need for repeat administration, and side effects.

Thus, there is the need for novel compositions and methods for the prevention and treatment of hot flashes, night sweats, and insomnia in symptomatic subjects.

### SUMMARY OF THE INVENTION

The inventor has discovered that scopolamine is efficacious in the treatment and prevention of hot flashes. In particular, it has been found that scopolamine, particularly when administered transdermally using a transdermal delivery device, diminishes the frequency and severity of hot flashes. A transdermal delivery device allows for a controlled release of the therapeutic agent, which thus avoids the problems of peaks and troughs of therapy associated with pill forms of therapy. Furthermore, the therapeutic window is narrowed, as there is not the need for passage of the therapeutic agent through the gastrointestinal tract and the liver.

The present invention generally pertains to methods of treating or preventing hot flashes, night sweats, and/or insomnia in a subject, comprising administering to the subject a therapeutically effective amount of scopolamine. Also disclosed are certain novel compositions of scopolamine that can be applied in the treatment and prevention of hot flashes, night sweats, and/or insomnia in a subject.

In certain embodiments of the present invention, administration of a therapeutic amount of scopolamine is further defined as placing a patch comprising a therapeutically effective amount of scopolamine on a surface of the subject, wherein delivery of scopolamine across the surface of the subject results in prevention or improvement of menopausal symptoms. For example, delivery may further be defined as transdermal delivery, transmucosal delivery, or transvaginal delivery. For example, the patch may be a transdermal delivery device for delivering a therapeutically effective amount of scopolamine to the subject.

Any subject is contemplated by the methods of the present invention. For example, the subject may be a mammal, such as a laboratory animal. In preferred embodiments, the subject is a human. The subject can be either male or female. For example, the subject may be a male or female with hot flashes or other symptoms due to hormonal changes.

In certain particular embodiments, the subject is further defined as a female with symptoms of hot flashes, night sweats, and/or insomnia related to menopause. One of ordinary skill in the art would be familiar with symptoms of menopause, which are discussed in greater detail in the specification below. The subject may also be a patient who has undergone chemotherapy and/or radiation therapy for the treatment of cancer, and wherein the patient is experiencing hot flashes, night sweats, and/or insomnia as a side effect of the chemotherapy and/or radiation therapy. For example, the cancer may be breast cancer, endometrial cancer, ovarian cancer, or prostate cancer.

In some embodiments, the subject is a patient who has undergone total abdominal hysterectomy with salpingo-oopherectomy, oopherectomy, or prostatectomy. The surgery could have been performed as treatment of any medical condition. For example, the subject may be a patient with cancer, and surgical treatment may have been performed as part of the therapeutic regimen. The cancer may be any type of cancer. For example, the cancer may be endometrial cancer or prostate cancer. Alternatively, the surgical therapy could have been performed for treatment of other disease. For example, hysterectomy with salpingo-oopherectomy may have been performed for treatment of uterine fibroids, endometriosis, and so forth.

The method may further comprise identifying a subject in need of treatment or prevention of hot flashes, night sweats, and/or insomnia. There are numerous ways in which one could identify a subject in need of treatment or prevention of hot flashes, night sweats, and/or insomnia, including interviewing a subject to identify a subject affected by or at risk of developing hot flashes, night sweats, or insomnia or completion of a questionnaire by a subject to identify a subject affected by or at risk of developing hot flashes, night sweats, or insomnia. The interview procedure may involve identification of those subjects who might be ineligible for estrogen therapy of hot flashes. For example, the subject may be ineligible for estrogen therapy because of a history of deep venous thrombosis, stroke, pulmonary embolism, myocardial infarction, endometrial carcinoma, or breast carcinoma.

In certain embodiments, the subject has hot flashes, and is a patient with breast cancer or a history of breast cancer who has undergone or is undergoing treatment with hormonal therapy, chemotherapy and/or radiation therapy. In other embodiments, the subject has hot flashes solely related to menopause.

Scopolamine can be delivered to the subject by any method known to those of ordinary skill in the art. Methods of administration of therapeutic agents are discussed in greater detail in the specification below. In certain preferred embodiments, the scopolamine is delivered via a patch. In particular embodiments, the patch is further defined as a transdermal delivery device. Patches and transdermal delivery devices are discussed in greater detail in the specification below.

For example, in some embodiments, the transdermal delivery device includes: (a) a backing layer; (b) a scopolamine reservoir; and (c) an adhesive layer. These components are discussed in greater detail in the specification below. For example, the transdermal delivery device may be any of the transdermal delivery devices set forth in U.S. Pat. No. 5,714,162 or U.S. Pat. No. 6,537,571, each of which is herein specifically incorporated by reference. Additional examples of transdermal delivery devices are discussed in the specification below.

In some embodiments, the transdermal delivery device further includes a membrane controlling the scopolamine flux from the patch to the surface of the subject. The scopolamine reservoir may be further defined as a pressure-sensitive scopolamine reservoir. In some embodiments, the transdermal delivery device further includes an additional skin adhesive layer. Adhesives are well-known to those of ordinary skill in the art, and are discussed in greater detail in the specification below. In some further embodiments, the transdermal delivery device further includes a peel strip and/or a protective film.

The scopolamine reservoir may further include any additional components or therapeutic agents. For example, the scopolamine reservoir may include a composition to increase saturation solubility of the scopolamine therein. Alternatively, the scopolamine reservoir may include a composition to decrease saturation solubility of the scopolamine therein.

Any dose of scopolamine is contemplated for treatment or prevention of the hot flashes, night sweats, and/or insomnia. Exemplary doses are set forth in the specification below. One of ordinary skill in the art would be familiar with methods and techniques to identify an optimum dosage for a particular subject. Various factors come into play, such as method of delivery of the therapeutic agent, associated side effects, and characteristics of the subject. These are discussed in greater detail in the specification below.

In certain particular embodiments wherein scopolamine is administered via a patch, the patch is formulated to include about 0.5 mg to about 5 mg of scopolamine. In certain more particular embodiments, the patch is formulated to include about 1.0 to about 3.0 mg scopolamine. In some even more particular embodiments, the patch includes about 1.5 mg scopolamine.

The dose of scopolamine can be repeated at any interval as determined by one of ordinary skill in the art for the treatment and prevention of hot flashes, night sweats, and/or insomnia. For example, in some embodiments, the patch is applied at a frequency of about every 3 days.

In some embodiments, one or more secondary therapies for hot flashes, night sweats, and/or insomnia is administered to the subject. For example, the secondary therapy may be a secondary therapy directed at preventing hot flashes, night sweats, and/or insomnia related to menopause. For example, the secondary therapy may be hormonal therapy. In certain particular embodiments, the hormonal therapy includes estrogen therapy, progesterone therapy, or a combination of estrogen and progesterone therapy. These forms of secondary therapy are discussed in greater detail in the specification below.

The secondary therapy may be formulated with scopolamine for delivery via a patch, such as a transdermal delivery device or other device designed for delivery of therapeutic agents across a skin or mucosal surface of a subject. The one or more additional therapeutic agents may be selected from the group consisting of an estrogen, a progesterone, and bellergal. In some embodiments, the transdermal delivery device is designed for delivery of bellergal, a composition that includes scopolamine, as discussed above. Exemplary estrogens include estrone, estradiol-17β, estriol, or a synthetic estrogen such as ethinylestradiol. Exemplary progesterones include progesterone or a synthetic progestin such as medroxyprogesterone, hydroxyprogesterone, medrogestone and norethindrone. One of ordinary skill in the art would be familiar with the various forms of estrogen, progesterone, and combinations thereof that can be applied in the treatment and prevention of hot flashes, night sweats, and/or insomnia in a subject.

In some embodiments of the methods set forth herein, scopolamine is administered in conjunction with one or more agents that counteract one or more side effects of scopolamine. One of ordinary skill in the art would be very familiar with the side effects of scopolamine. Examples include drowsiness, dry mouth, and dizziness. In certain embodiments, the secondary agent is formulated in a patch with the scopolamine, as discussed above. Exemplary secondary agents to counteract one or more side effects of scopolamine are discussed in greater detail in the specification below. In other embodiments, bellergal, a formulation that includes scopolamine, is formulated in a patch.

As used herein the specification, “a” or “an” may mean one or more. As used herein in the claim(s), when used in conjunction with the word “comprising”, the words “a” or “an” may mean one or more than one. As used herein “another” may mean at least a second or more.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### DESCRIPTION OF ILLUSTRATIVE EMBODIMENTS

The present invention is based on the surprising discovery that scopolamine is beneficial in reducing hot flashes, night sweats, and insomnia. The hot flashes, night sweats, and insomnia can be of any etiology. For example, it has been discovered that transdermal administration of scopolamine is beneficial in reducing hot flashes that are caused by treatment of breast cancer with hormonal therapy, chemotherapy and/or radiation therapy. The present invention also concerns combinations of scopolamine with hormonal therapy for transdermal delivery via a patch. A detailed description of the methods and compositions of the present invention is set forth as follows.

### A. SCOPOLAMINE

1. Definition of Scopolamine

Scopolamine is α-(hydroxymethyl) benzeneacetic acid 9-methyl-3-oxa-9-azatricyclo [3.3.1.0] non-7-yl ester (discussed in detail in Gilman et al., 1990 and Kotelko et al., 1989, each of which is herein incorporated by reference in its entirety). Scopolamine is a viscous liquid that has a molecular weight of 303.35 and a pKa of 7/55-7.81. It is a belladonna alkaloid with well-known pharmacological properties (Gilman et al., 1990 and Kotelko et al., 1989).

“Scopolamine,”as the term is used herein, refers both to (+)-scopolamine and (−)-scopolamine, or racemic mixtures of both, or salts thereof. (+)-Scopolamine, a heterocyclic compound with a single chiral center to which a MeOH group is bonded, exists as an enantiomer of hyoscine ((−)-scopolamine) (Glasby, 1975). Biologically, both forms of scopolamine have similar function and activity although they are chemically distinct. The two chemical forms may be separated by chromatography (Fodor, 1957), or based on melting points of crystallized salts and show the same ¹³C-NMR and H-NMR.

“Scopolamine,” as the term is used herein, also includes any substituted variant of scopolamine, or a salt of scopolamine. Because of its many exposed functional groups, scopolamine is readily subject to chemical and biological decomposition and is thus stored and administered as a hydrated hydro-halogen (typically HCl or HBr) salt for stability. The molecule shows weak acidic properties at its tropic acid group while the methylated nitrogen on the tropane ring readily accepts a hydrogen. Synthetically and naturally, the tropic acid group is added as a complete unit in a state very similar to its final form due to the relative ease of esterification of tropic carbon 3, and due to the stability of the heterocyclic tropane system.

As discussed above, scopolamine and one of its salts, scopolamine hydrogen-bromide, have found wide use and acceptance for use as anti-motion-sickness agents. The drug itself may be ingested, injected intravenously or applied topically as a patch behind the ear. After rapid absorption, scopolamine is excreted via urine after nearly complete metabolism in the liver. Side effects of scopolamine can include dry mouth, drowsiness, and blurred vision.

“Scopolamine,” as used herein, also includes derivatives of scopolamine, such as those set forth in U.S. Pat. No. 3,952,108 and U.S. Pat. No. 3,767,786, each of which is herein specifically incorporated by reference.

2. Source of Scopolamine

Scopolamine can be extracted from natural sources, such as from the mother liquor extracts of the plant Hyoseyamus (www.ucalgary.ca/bali/chem.353/project/00final/00final.htm) or chemically synthesized, such as from 3α-acetoxytrop-6-ene, obtained via 3 routes, through several alkaloid intermediates (Fodor, 1957).

The sites of synthesis and accumulation of scopolamine differ from plant to plant within the genus Datura. In general, scopolamine is synthesized in the roots of young plants and accumulates in the aerial parts (Marion and Thomas, 1955). Using carbon and hydrogen isotopes, Romeik and Aurich were among those who discovered the method by which scopolamine is synthesized (Conklin, 1976).

Biosynthesis is regulated by numerous enzymes in the plant, including tropinone reductase I and hyoscyamine 6β-hydroxylase. The actual synthesis of scopolamine follows a tropinone pathway, which also generates many other products. The amino acid ornithine is converted to N-methylornithine, which is a precursor to N-methylputrescine, via putrescine N-methyl transferase (Leete et al., 1954).

### B. DISEASES AND CONDITIONS TO BE TREATED AND PREVENTED

As used herein, “treating” refers to administration of a therapeutic agent or therapeutic modality for the purpose of eliminating or diminishing the frequency or severity of a disease or health-related condition in a subject. In the context of the present invention, “treating” pertains to administration of one or more therapeutic agents, one of which is scopolamine, for the purpose of eliminating or diminishing the frequency or severity of hot flashes, night sweats, and/or insomnia.

“Preventing” refers to the administration of a therapeutic agent or therapeutic modality for the purpose of blocking the onset of a disease or health-related condition. In the context of the present invention, “preventing” pertains to the administration of one or more therapeutic agents, one of which is scopolamine, for the purpose of blocking the onset of hot flashes, night sweats, or insomnia.

The methods and compositions of the present invention can be applied in the treatment and prevention of hot flashes, night sweats, and insomnia of any cause. “Hot flashes,” as defined above, refers to a feeling of warmth, sometimes associated with flushing, that spreads over the body and may be accompanied by perspiration. They can occur with varying frequency during the course of a day or night.

There are various causes of hot flashes. Perhaps the most common cause is menopause (or the climacteric). Menopause is defined as 12 months without a menstrual period in the absence of another pathological or physiologic cause. It is the cessation of ovarian function and is associated with low estradiol levels and as a result elevated FSH levels. As the term is used herein, “menopause” also includes the perimenopause, which is a period of waxing and waning ovarian function where normal cycles are interspersed with anovulatory cycles.

The symptoms of menopause, including the vasomotor symptoms of hot flashes and night sweats, are generally believed to be related to estrogen deficiency. Other symptoms of menopause include vaginal dryness, frequent urinary tract infections, and incontinence. Depressed mood, decreased libido and sleep disturbance are also commonly attributed to menopause although it is unclear if these are truly symptoms related to estrogen deficiency or if they arise from coping with the other symptoms and stresses related to menopause.

The average age of menopause is 51, but in smokers it is, on average, 2 years earlier. Only 1% of women become postmenopausal before age 40, and 5% become post menopausal after the age of 55.

Other causes of hot flashes include hot flashes triggered by hormonal therapy, chemotherapy and/or radiation therapy in pre-menopausal and post-menopausal breast cancer survivors. Hot flashes can also be present in patients who have undergone hysterectomy or oopherectomy as a result of fluctuation in hormone levels. The hysterectomy and oopherectomy can be of any cause, such as benign tumors (e.g., uterine fibroids), malignancy (e.g., cervical carcinoma, vaginal carcinoma, endometrial carcinoma, ovarian carcinoma, metastatic disease), or other conditions (e.g., endometriosis).

Hot flashes can also occur in men who have a history of prostate cancer, as discussed above. The exact cause is unknown.

Hot flashes may be associated with other symptoms in menopausal women, including night sweats and insomnia. The insomnia that often occurs in women with menopause may or may not be aggravated by night sweats and/or hot flashes.

“Night sweats,” as defined herein, refers to a period of excessive sweating, usually occurring during the night while the subject is sleeping. It is not uncommon for the patient to awake from sleep drenched in sweat. A “night sweat” can also be the result of a “hot flash” occurring at night while the subject is sleeping. The methods of treatment and compositions of the present invention can be applied in the treatment and prevention of night sweats of any cause.

Night sweats and other vasomotor symptoms are a common outpatient complaint. Hot flashes and night sweats are common during menopause but many women experience them before they cease menstrual function for a year and become menopausal. Night sweats are also a common complaint of women with artificial menopause who have undergone chemotherapy and/or radiation therapy for treatment of breast cancer or endometrial cancer, as well as women who have undergone hysterectomy and/or oopherectomy.

Other causes of night sweats include diseases such as infectious diseases (e.g., tuberculosis, HIV), malignancies (e.g., lymphoma), gastroesophageal reflux disease, obstructive sleep apnea, hyperthyroidism, hypoglycemia, and several less common diseases. Antihypertensives, antipyretics, other medications, and drugs of abuse such as alcohol and heroin may cause night sweats.

“Insomnia” is defined herein to refer to an inability to sleep and/or to remain asleep for a reasonable period of time. The methods and compositions of the present invention can be applied in the treatment and prevention of insomnia of any cause. Insomnia is a common complaint in patients experiencing hot flashes and night sweats. Insomnia or sleeplessness is a common complaint of women as they enter into menopause. For patients who are having hot flashes or night sweats at night, called night sweats, their sleep may be broken by frequent awakening, thus resulting in insomnia.

### C. PATCHES, TRANSDERMAL DELIVERY DEVICES, AND OTHER DELIVERY DEVICES

1. Patches

As used herein, a “patch” is a material or covering that can be applied to a surface of the body. For example, the surface can be a skin surface or a mucosal surface (e.g., the surface of the vagina or mouth). In certain embodiments of the present invention, the patch can include scopolamine applied to one or more surfaces of the patch. Furthermore, one or more additional therapeutic agents for the treatment of hot flashes, night sweats, and insomnia can be included. In some embodiments, one or more additional therapeutic agents for the treatment and/or prevention of side effects of scopolamine treatment can be included with the scopolamine.

The patch can be composed of any material known to those of ordinary skill in the art. Further, the patch can be designed for delivery of the therapeutic agent(s) by application of the patch to a body surface of a subject, such as a skin surface, the surface of the oral mucosa, a wound surface, or the surface of a tumor bed. The patch can be designed to be of any shape or configuration, and can include, for example, a strip, a bandage, a tape, a dressing (such as a wound dressing), or a synthetic skin.

2. Transdermal Delivery Devices

A “transdermal delivery device” is defined herein to refer to a patch comprising a therapeutic agent that can be applied to the surface of the skin for the purpose of transdermally delivering a therapeutic agent. For example, the transdermal delivery device can include a patch (or backing layer), a reservoir to include the active agents, and optionally an adhesive layer. Adhesive layers are discussed in greater detail below. Alternatively, the active substance may be comprised in a plurality of microcapsules which are distributed within a permeable adhesive layer. In any case, the active substances are continuously released from the reservoir or microcapsules through a membrane into the adhesive layer which is permeable to the active substances and which is in contact with the skin or mucosa of the person to be treated. In the case of microcapsules, the material of the capsule may also act as a membrane.

In some embodiments, the device may be designed with a membrane to control the rate at which a liquid or semi-solid formulation of the therapeutic agent can pass through the skin and into the blood stream. Components of the device may include, for example, the therapeutic agent dissolved or dispersed in a reservoir or inert polymer matrix; an outer backing film of paper, plastic, or foil; and a pressure-sensitive adhesive that anchors the patch to the skin. The adhesive may or may not be covered by a release liner, which needs to be peeled off before applying the patch to the skin. In some embodiments, the therapeutic agent is contained in a hydrogel matrix. In other embodiments, the patch is designed to use a low power electric current to transport the therapeutic agent through the skin. In other embodiments, the patch is designed for passive drug transport through the skin or mucosa. In other embodiments, the device is designed to utilize iontophoresis for delivery of the therapeutic agent.

The device may include a reservoir wherein the therapeutic agent is comprised in a solution or suspension between the backing layer and a membrane that controls the rate of delivery of the therapeutic agent. In other embodiments, the device includes a matrix comprising the therapeutic agent, wherein the therapeutic agent is in a solution or suspension dispersed within a collagen matrix, polymer, or cotton pad to allow for contact of the therapeutic agent with the skin. In some embodiments, an adhesive is applied to the outside edge of the delivery system to allow for adhesion to a surface of the subject.

In some embodiments, the device is composed of a substance that can dissolve on the surface of the subject following a period of time. For example, the device may be a film or skin that can be applied to the mucosal surface of the mouth, wherein the device dissolves in the mouth after a period of time. The therapeutic agent, in these embodiments, may be either applied to a single surface of the device (i.e., the surface in contact with the subject), or impregnated into the material that composes the device.

In some embodiments, the device is designed to incorporate more than one therapeutic agent. In these embodiments, the device may comprise separate reservoirs for each therapeutic agent, or may contain multiple therapeutic agents in a single reservoir. As discussed above, the additional therapeutic agent or agents may be an additional therapeutic agent for the treatment or prevention of hot flashes, night sweats, and/or insomnia in a subject. The additional therapeutic agent or agents may be a therapeutic agent to counteract the side effects of scopolamine therapy.

In some embodiments, the device is designed to vary the rate of delivery of the therapeutic agent based on bodily changes in the subject, such as temperature or perspiration. For example, certain agents may be comprised in a membrane covering the therapeutic agent that respond to temperature changes and allow for varying levels of drug to pass through the membrane. In other embodiments, transdermal or transcutaneous delivery of the therapeutic agent can be varied by varying the temperature of the patch through incorporation of a temperature-control device into the device.

One of ordinary skill in the art would be familiar with methods and techniques for transdermal and transcutaneous delivery of drugs using patches. Devices and techniques for transdermal delivery of scopolamine are discussed in greater detail in U.S. Pat. No. 6,537,571, and U.S. Pat. No. 5,939,095, both of which are herein specifically incorporated by reference in their entirety for this and all sections of this specification. Additional information pertaining to transdermal delivery devices can be found in U.S. Pat. No. 6,818,226, U.S. Pat. No. 6,365,178, U.S. Pat. No. 6,299,900, U.S. Pat. No. 5,662,928, U.S. Pat. No. 5,260,066, U.S. Pat. No. 4,938,759, U.S. Pat. No. 4,917,676, U.S. Pat. No. 4,865,846, U.S. Pat. No. 4,837,027, U.S. Pat. No. 4,781,924, U.S. Pat. No. 4,559,222, U.S. Pat. No. 3,993,072, U.S. Pat. No. 3,962,414, U.S. Patent App. Pub. No. 20040018241, U.S. Patent App. Pub. No. 20050129749, U.S. Patent App. Pub. No. 20060078601, U.S. Patent App. Pub. No. 20060078602, U.S. Patent App. Pub. No. 20060078603, and U.S. Patent App. Pub. No. 20060078604, U.S. Pat. No. 4,559,222, U.S. Pat. No. 4,781,924, U.S. Pat. No. 4,837,027, U.S. Pat. No. 4,904,475, U.S. Pat. No. 4,938,759, and U.S. Pat. No. 5,167,616, each of which is herein specifically incorporated by reference in their entirety.

3. Other Delivery Devices

Scopolamine may be administered by other delivery devices, including, but not limited to, transmucosal delivery devices. These devices may take the form of patches or strips that can be applied to the surface of the oral cavity. For example, the patches or strips may, in certain embodiments, be designed to dissolve over a period of time. Oral dissolvable medicaments are discussed in greater detail in U.S. Pat. No. 5,785,989, U.S. Pat. No. 5,288,498, and U.S. Pat. No. 5,288,497, each of which is herein specifically incorporated by reference in its entirety. In other embodiments, the scopolamine is delivered via a vaginal insert, such as a silastic ring. Vaginal drug delivery devices are discussed in greater detail in U.S. Pat. No. 6,899,700, U.S. Pat. No. 6,758,840, U.S. Pat. No. 6,888,043, U.S. Pat. No. 6,805,877, U.S. Pat. No. 6,669,703, Englund and Johansson, 1978, and Englund et al., 1981, each of which is herein specifically incorporated by reference in its entirety. Interlabial pads for delivery of therapeutic agents, also contemplated by the present invention, is discussed in greater detail in U.S. Pat. No. 6,811,549, which is herein incorporated by reference in its entirety.

4. Adhesives

Certain embodiments of the present invention pertain to transdermal delivery devices and other delivery devices comprising an adhesive.

Adhesives for use in pharmaceutics and medicine are well-known to those of ordinary skill in the art, and include topical skin adhesives such as sterile, liquid glue, as well as solid or semi-solid adhesives. Adhesives for use in the present invention also include adhesives that are liquid upon application, but which rapidly dry to a solid consistency.

Exemplary adhesives for use in the compositions and methods of the present invention include acrylates, such as cyanoacrylate, methacrylates, and alkyl acrylates. Other exemplary adhesives include hydrocolloids, hydrogels, polyisobutylene, and adhesives that are based on a gel matrix, such as polyacrylic acid-based gel matrix adhesives.

Tissue adhesives are also contemplated for use in the pharmaceutical compositions and methods of the present invention. Compositions pertaining to tissue adhesives are discussed in detail in U.S. Patent Appn. 20040199207, U.S. Patent Appn. 20030119985, U.S. Patent Appn. 20020116026, U.S. Patent Appn. 20020037323, U.S. Pat. No. 6,723,114, U.S. Pat. No. 6,596,318, U.S. Pat. No. 6,329,337, U.S. Pat. No. 6,310,036, U.S. Pat. No. 6,299,631, and U.S. Pat. No. 6,251,370, each of which is herein specifically incorporated by reference.

### D. DOSAGE

An effective amount of scopolamine and any other therapeutic or preventive agent is determined based on the intended goal, for example (i) decrease in the frequency or severity of hot flashes or (ii) prevention of hot flashes.

Those of skill in the art are well aware of how to deliver therapeutic agents for treatment of a disease or health-related condition. Formulation as a pharmaceutically acceptable composition is discussed below.

The dose to be administered, both according to number of treatments and dose, depends on the subject to be treated, the state of the subject and the protection desired. Precise amounts of the therapeutic composition also depend on the judgment of the practitioner and are peculiar to each individual.

For example, the dose may be about 0.1 mg to about 10 mg, or any dose range derivable therein. For example, the dose may be about 0.2 mg., about 0.25 mg, about 0.30 mg, about 0.35 mg, about 0.4 mg, about 0.45 mg, about 0.5 mg, about 0.55 mg, about 0.60 mg, about 0.65 mg, about 0.7 mg, about 0.75 mg, about 0.80 mg, about 0.85 mg, about 0.90 mg, about 0.95 mg, about 1.0 mg, about 1.05 mg, about 1.1 mg, about 1.15 mg, about 1.20 mg, about 1.25 mg, about 1.30 mg, about 1.35 mg, about 1.40 mg, about 1.45 mg, about 1.50 mg, about 1.55 mg, about 1.60 mg, about 1.65 mg, about 1.70 mg, about 1.75 mg, about 1.8 mg, about 1.85 mg, about 1.9 mg, about 1.95 mg, about 2.0 mg, about 2.1 mg, about 2.2 mg, about 2.3 mg, about 2.4 mg, about 2.5 mg, about 2.6 mg, about 2.7 mg, about 2.8 mg, about 2.9 mg., about 3.0 mg, about 3.1 mg, about 3.2 mg, about 3.3 mg, about 3.4 mg, about 3.5 mg, about 3.6 mg, about 3.7 mg, about 3.8 mg, and 3.9 mg, about 4.0 mg, about 4.1 mg, about 4.2 mg, about 4.3 mg, about 4.4 mg, about 4.5 mg, about 4.6 mg, about 4.7 mg, about 4.8 mg, about 4.9 mg, about 5 mg, about 5.5 mg, about 6.0 mg, about 6.5 mg, about 7.0 mg, about 7.5 mg, about 8.0 mg, about 8.5 mg, about 9.0 mg, about 9.5 mg, about 10.0 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 35 mg, about 40 mg, about 45 mg, about 50 mg, about 60 mg, about 70 mg, about 80 mg, about 90 mg, about 100 mg, our any higher amount of scopolamine, or any intermediate amount of scopolamine between any of the listed amounts set forth herein, or any dose range derivable therein.

In some embodiments, it may be desirable to provide a continuous supply of the therapeutic compositions to the patient. For topical administrations, administration via a delivery device, such as a transdermal delivery device, discussed in greater detail below, would facilitate controlled release over a prolonged period of time.

The dose of scopolamine (e.g., replacement of the patch) can be repeated as needed as determined by those of ordinary skill in the art. Dosing interval is dependent upon a number of factors, such as response to therapy, side effects, and so forth. For example, if scopolamine is delivered via a transdermal delivery device, the patch may be replaced about every 1-5 days, or in certain embodiments, every three days.

### E. PHARMACEUTICAL COMPOSITIONS

1. Definitions

Certain preferred embodiments of the present invention pertain to pharmaceutical compositions comprising a therapeutically effective amount of scopolamine. “Pharmaceutical compositions” refers to molecular entities and compositions that do not produce an adverse, allergic or other untoward reaction when administered to a mammal or human, as appropriate. A “therapeutically effective amount” of scopolamine is an amount of scopolamine that is known or suspected to be on benefit in treating or preventing hot flashes, night sweats, and/or insomnia in a subject. For example, a “therapeutically effect amount” of scopolamine can be a dose of scopolamine that is known or suspected to be of benefit in reducing the frequency or severity of hot flashes in a subject.

Pharmaceutical compositions includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients also can be incorporated into the composition. In addition, the composition can include supplementary inactive ingredients, such as binding agents and coloring agents.

In some instances, the pharmaceutical composition is formulated for topical delivery or topical administration. In the context of the present invention, “topical administration” is defined to include administration to a surface of the body such as the skin, oral mucosa, cervix, vagina, anus, eye, or administration to the surface of the bed of an excised lesion in any of these areas.

In further embodiments of the present invention, the pharmaceutical composition is formulated for oral delivery. Oral delivery includes administration via the mouth of an animal or other mammal, as appropriate. Oral delivery includes topical administration to any part of the oral cavity.

2. Solid or Semi-Solid Formulations

Certain embodiments of the present invention pertain to pharmaceutical compositions of scopolamine that is formulated in a solid form or semi-solid form. One of ordinary skill in the art would be familiar with formulation of agents as a solid or semi-solid.

For example, the solid may be a gel, a matrix, a foam, a cream, an ointment, a lozenge, a lollipop, a gum, a powder, a troche, a gel strip, a film, a hydrogel, a dissolving strip, a paste, a toothpaste, or a solid stick.

Methods pertaining to the formulation of creams are set forth in U.S. Pat. No. 6,620,451, U.S. Pat. No. 6,261,574, U.S. Pat. No. 5,874,094, and U.S. Pat. No. 4,372,944, each of which is herein specifically incorporated by reference in its entirety. Methods pertaining to the formulation of gels are set forth in U.S. Pat. No. 6,280,752, U.S. Pat. No. 6,258,830, U.S. Pat. No. 5,914,334, U.S. Pat. No. 5,888,493, and U.S. Pat. No. 5,571,314, each of which is herein specifically incorporated by reference in its entirety. Methods pertaining to the formulation of ointments are set forth in U.S. Pat. No. 5,078,993, U.S. Pat. No. 4,868,168, and U.S. Pat. No. 4,526,899, each of which is herein specifically incorporated by reference in its entirety. Methods pertaining to the formulation of pastes are set forth in U.S. Pat. No. 4,627,979, U.S. Pat. No. 6,508,647, U.S. Patent Appn. 20020045148, and U.S. Patent Appn. 20040018155, each of which is herein specifically incorporated by reference in its entirety

In particular embodiments, oral pharmaceutical compositions will comprise an inert diluent and/or assimilable edible carrier, and/or they may be enclosed in hard and/or soft shell gelatin capsule, and/or they may be compressed into tablets, and/or they may be incorporated directly with the food of the diet. For oral therapeutic administration, the active compounds may be incorporated with excipients and/or used in the form of ingestible tablets, buccal tables, troches, capsules, elixirs, suspensions, syrups, wafers, and/or the like.

Solid forms suitable for solution in, or suspension in, liquid prior to topical use are also contemplated by the present invention.

The solid and semisolid formulations of the present invention may contain the following: a binder, as gum tragacanth, acacia, cornstarch, and/or gelatin; excipients, such as dicalcium phosphate; a disintegrating agent, such as corn starch, potato starch, alginic acid and/or the like; a lubricant, such as magnesium stearate; a fragrance, and/or a sweetening agent, such as sucrose, lactose and/or saccharin may be added and/or a flavoring agent, such as peppermint, oil of wintergreen, and/or cherry flavoring. When the dosage unit form is a capsule, it may contain, in addition to materials of the above type, a liquid carrier. Various other materials may be present as coatings and/or to otherwise modify the physical form of the dosage unit. For instance, tablets, pills, and/or capsules may be coated with shellac, sugar and/or both. Preservatives, dyes, and flavorings known to those of ordinary skill in the art are contemplated.

Additional formulations which are suitable for other modes of administration include vaginal suppositories and/or pessaries. A rectal pessary and/or suppository may also be used. Suppositories are solid dosage forms of various weights and/or shapes, usually medicated, for insertion into the rectum, vagina and/or the urethra. After insertion, suppositories soften, melt and/or dissolve in the cavity fluids. In general, for suppositories, traditional binders and/or carriers may include, for example, polyalkylene glycols and/or triglycerides; such suppositories may be formed from mixtures containing the active ingredient in the range of 0.5% to 10%, preferably 1%-2%.

Formulations for other types of administration that is topical include, for example, a cream, suppository, ointment or salve. One of ordinary skill in the art would be familiar with formulation of therapeutic agents as a cream, suppository, ointment, or salve.

In some embodiments, the active ingredient may be added in a therapeutically effective amount to a paste dentifrice that may include water, binders, abrasives, flavoring agents, foaming agents, and humectants.

Formulations designed to be administered via patches and transdermal delivery devices are discussed in detail elsewhere in this specification.

3. Aqueous Formulations

Certain of the therapeutic compositions of the present invention can be formulated as aqueous compositions. Aqueous compositions of the present invention comprise an effective amount of scopolamine, dissolved or dispersed in a pharmaceutically acceptable carrier or aqueous medium.

Examples of aqueous compositions include a mouthwash, mouthrinse, a liquid, or a liquid suitable for aerosol administration. Mouthwash formulations are well-known to those of skill in the art. Formulations pertaining to mouthwashes and oral rinses are discussed in detail, for example, in U.S. Pat. No. 6,387,352, U.S. Pat. No. 6,348,187, U.S. Pat. No. 6,171,611, U.S. Pat. No. 6,165,494, U.S. Pat. No. 6,117,417, U.S. Pat. No. 5,993,785, U.S. Pat. No. 5,695,746, U.S. Pat. No. 5,470,561, U.S. Pat. No. 4,919,918, U.S. Patent Appn. Pub. No. 20040076590, U.S. Patent Appn. Pub. No. 20030152530, and U.S. Patent Appn. Pub. No. 20020044910, each of which is herein specifically incorporated by reference into this section of the specification and all other sections of the specification. Other examples of aqueous compositions include a douche solution for vaginal use, spray or aerosol, or ophthalmic solution.

Solutions of the active compounds as free base or pharmacologically acceptable salts can be prepared in water suitably mixed with a surfactant, such as hydroxypropylcellulose. Dispersions also can be prepared in glycerol, liquid polyethylene glycols, mixtures thereof and in oils. Under ordinary conditions of storage and use, these preparations contain a preservative to prevent the growth of microorganisms.

Administration of certain embodiments of the pharmaceutical compositions set forth herein will be via any common route so long as the target tissue is available via that route. For example, this includes oral, nasal, buccal, anal, rectal, vaginal, or topical ophthalmic. Such compositions would normally be administered as pharmaceutically acceptable compositions that include physiologically acceptable carriers, buffers or other excipients. Examples of other excipients include fragrances and flavorants.

The formulation may be in a liquid form or suspension. A typical composition for such purpose comprises a pharmaceutically acceptable carrier. For instance, the composition may contain 10 mg, 25 mg, 50 mg or up to about 100 mg of human serum albumin per ml of phosphate buffered saline. Other pharmaceutically acceptable carriers include aqueous solutions, non-toxic excipients, including salts, preservatives, buffers and the like. Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Preservatives include antimicrobial agents, anti-oxidants, chelating agents and inert gases. The pH and exact concentration of the various components of the pharmaceutical composition are adjusted according to well-known parameters.

Oral formulations include such normally employed excipients as, for example, pharmaceutical grades of mannitol, lactose, starch, magnesium stearate, sodium saccharine, cellulose, magnesium carbonate and/or the like. Such compositions and/or preparations should contain at least 0.1% of active compound. The percentage of the compositions and/or preparations may, of course, be varied and/or may conveniently be between about 2 to about 75% of the weight of the unit, and/or preferably between 25-60%. The amount of active compounds in such therapeutically useful compositions is such that a suitable dosage will be obtained.

One may also use solutions and/or sprays, hyposprays, aerosols and/or inhalants in the present invention for administration. The sprays are isotonic and/or slightly buffered to maintain a pH of 5.5 to 6.5. In addition, antimicrobial preservatives, similar to those used in ophthalmic preparations, and/or appropriate drug stabilizers, if required, may be included in the formulation.

4. Formulations with One or More Agents to Counteract Adverse Side Effects of Scopolamine

In some embodiments of the present invention, scopolamine is formulated with one or more secondary agents to counteract any unwanted side effects of scopolamine. These side effects include, but are not limited to, dry mouth, drowsiness, transient impairment of eye accomodation, blurred vision, and dizziness. In these embodiments, the secondary agent may be formulated with the scopolamine (such as a transdermal delivery device comprising scopolamine and one or more secondary agent to counteract drowsiness). For example, the second agent may be caffeine or any other agent to prevent drowsiness.

The second agent may also be an agent to prevent dry mouth. Agents to prevent dry mouth are well-known to those of ordinary skill in the art. Examples include saliva substitutes such as Salivart® oral moisturizer or a biotin-containing oral moisturizer such as Biotene mouthwash. Other examples include pilocarpine or cevimeline, delivered orally. If scopolamine is administered via an oral transmucosal delivery device, for example, the scopolamine may be formulated with a wetting agent, many of which are well-known to those of ordinary skill in the art.

In certain embodiments of the methods of the present invention, the secondary agent to counteract an unwanted side effect of scopolamine may be administered separately from the scopolamine. For example, administration of scopolamine via a transdermal delivery device combined with oral delivery of caffeine to prevent drowsiness.

### F. SECONDARY THERAPIES

In particular embodiments, the scopolamine is administered with one or more secondary forms of therapy for the prevention or treatment of hot flashes, night sweats, and/or insomnia.

A wide variety of secondary therapies, known to one of skill in the art, may be used in combination with the compositions of the claimed invention. One of ordinary skill in the art would be familiar with the range of agents that can be applied as secondary therapies in the treatment of hot flashes, night sweats, and/or insomnia in a subject. For example, scopolamine may be formulated with one or more forms of hormonal therapy for hot flashes for delivery via a transdermal delivery device. For example, the hormonal therapy may be an estrogen, a progesterone, or a combination of an estrogen and a progesterone. Administration of the hormonal therapy in conjunction with scopolamine may allow for lower doses of hormonal therapy, and thus lower risk of untoward side effects of hormonal therapy.

Regarding estrogens, the estrogens may either contain a “single” “estrogen component (e.g. Premarin) or a combination of an estrogen and a progestin (e.g. Prempro or Activella) or an estrogen and an androgen (e.g. Estradiol/M-testosterone). For example, Premarin is a mixture of weak estrogens—containing estrone, equilin, and 17 α-dihydroequilin, 17 α-estradiol, equilenin, and 17 α-dihydroequilenin as salts of their sulfate esters. Equilin (and metabolites) is an estrogen-like hormone derived from horses.

The estrogen may also be a conjugated estrogen. A conjugated estrogen is either a mixture of different natural weak estrogens converted to a water-soluble form, (e.g. sulfates as in Premarin), or a combination of estrogens with other compounds (e.g. Estradiol cypionate). Examples of conjugated estrogens include estrogen/medroxyprogesterone, stradial/norethindrone acetate, estradiol/M-testosterone, conjugated estrogen/meprobamat, conjugated estrogen/methyltestosterone, piperazine estrone sulfate (estropipate), estradiol cypionate, estradiol valerate, estrogens esterified, and estrone estradiol vaginal tablets.

The estrogen may also be a phytoestrogen. Phytoestrogens are plant-derived compounds that are structurally similar to the natural human estrogen, estradiol-17β. Most of these compounds are classified as isoflavones and the most recognized members of this group of estrogens are daidzein and genistein, which are the dominant species found in soy. Phytoestrogens are the principal constituents of Estroven®.

One of ordinary skill in the art would be familiar with progesterones. Exemplary progesterones include medroxyprogesterone or norethindrone acetate.

Alternatively, the secondary form of therapy may be administered separately from scopolamine. Scopolamine therapy may precede or follow the other agent treatment by intervals ranging from minutes to weeks. In embodiments where the other agent and scopolamine are applied separately to the cell, one would generally ensure that a significant period of time did not expire between the time of each delivery, such that the scopolamine and second agent would still be able to exert an advantageously combined effect on the subject. In such instances, it is contemplated that one may administer scopolamine and treat with the secondary agent either concurrently, or within days or weeks of each other.

### G. EXAMPLES

### Example 1

### Transdermal Administration of Scopolamine Eliminates Hot Flashes

A subject, specifically a post-menopausal breast cancer survivor with a long history of hot flashes, provided proof of this invention. Hormonal therapy of hot flashes was contraindicated because of the history of breast cancer. Retroauricular placement of one-half of a 1.5 mg transdermal scopolamine patch (Transderm Sc o̅p̅, ALZA Corp., Mountain View, Calif.), with replacement of the patch every three days, was found to eliminate hot flashes in the subject. Similar results were obtained in another subject with symptoms of hot flashes related to menopause, with no history of breast cancer.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 3,767,786
U.S. Pat. No. 3,952,108
U.S. Pat. No. 3,962,414
U.S. Pat. No. 3,993,072
U.S. Pat. No. 4,372,944
U.S. Pat. No. 4,526,899
U.S. Pat. No. 4,559,222
U.S. Pat. No. 4,627,979
U.S. Pat. No. 4,781,924
U.S. Pat. No. 4,837,027
U.S. Pat. No. 4,865,846
U.S. Pat. No. 4,868,168
U.S. Pat. No. 4,917,676
U.S. Pat. No. 4,919,918
U.S. Pat. No. 4,938,759
U.S. Pat. No. 5,078,993
U.S. Pat. No. 5,288,497
U.S. Pat. No. 5,288,498
U.S. Pat. No. 5,360,066
U.S. Pat. No. 5,470,561
U.S. Pat. No. 5,571,314
U.S. Pat. No. 5,662,928
U.S. Pat. No. 5,695,746
U.S. Pat. No. 5,714,162
U.S. Pat. No. 5,785,989
U.S. Pat. No. 5,874,094
U.S. Pat. No. 5,888,493
U.S. Pat. No. 5,914,334
U.S. Pat. No. 5,939,095
U.S. Pat. No. 5,993,785
U.S. Pat. No. 6,117,417
U.S. Pat. No. 6,165,494
U.S. Pat. No. 6,171,611
U.S. Pat. No. 6,251,370
U.S. Pat. No. 6,258,830
U.S. Pat. No. 6,261,574
U.S. Pat. No. 6,280,752
U.S. Pat. No. 6,299,631
U.S. Pat. No. 6,299,900
U.S. Pat. No. 6,310,036
U.S. Pat. No. 6,329,337
U.S. Pat. No. 6,348,187
U.S. Pat. No. 6,365,178
U.S. Pat. No. 6,387,352
U.S. Pat. No. 6,508,647
U.S. Pat. No. 6,537,571
U.S. Pat. No. 6,537,571
U.S. Pat. No. 6,596,318
U.S. Pat. No. 6,620,451
U.S. Pat. No. 6,669,703
U.S. Pat. No. 6,723,114
U.S. Pat. No. 6,758,840
U.S. Pat. No. 6,805,877
U.S. Pat. No. 6,811,549
U.S. Pat. No. 6,818,226
U.S. Pat. No. 6,888,043
U.S. Pat. No. 6,899,700
U.S. Patent Appn. 20020037323
U.S. Patent Appn. 20020044910
U.S. Patent Appn. 20020045148
U.S. Patent Appn. 20020116026
U.S. Patent Appn. 20030119985
U.S. Patent Appn. 20030152530
U.S. Patent Appn. 20040018155
U.S. Patent Appn. 20040076590
U.S. Patent Appn. 20040199207
Chlebowski et al., JAMA, 289(24):3243-3253, 2003.
Conklin, London: S. Karger, 1(3):118-122, 1976.
Englund and Johansson, Brit. J Obstet. Gynecol., 85:957, 1978.
Englund et al., Maturitas, 3(2):125-133, 1981.
Fodor, Tetrahedron, 14:86, 1957.
Freedman and Blacker, Fertil. Steril., 77(3):487-490, 2002.
Gilman et al., In: The Pharmacological Basis of Therapeutics, 8^{th} Ed., Pergamon Press, NY, 150-165, 1990.
Kotelko et al., Anesthesiology, 71(5):675-678, 1989.
Lagrelius et al., Acta Obstet. Gynecol. Scand., 60(1):27-31, 1981.
Lette et al., Nature, 174:650, 1954.
Marion and Thomas, Can. J. Chem., 33:1853, 1955.
McEvoy, In: AHSF Drug Information, Amer. Soc. Hosp. Pharm., Bethesda, Md., 608-611, 1990.
Molina et al., Drug Saf., 28(5):401-416, 2005.
Mourits et al., Obstet Gynecol. 97(5 Pt 2):855-66, 2001.
Pritchard, Oncologist, 6(4):353-362, 2001.
Spetz et al., J Support Oncol., 1(4):263-266; 269-270; 272-273, 2003.
Writing Group for the Women's Health Initiative Investigators, JAMA, 288(3):321-333, 2002.

What is claimed is:

1. A method of treating or preventing hot flashes, night sweats, and/or insomnia in a subject, comprising administering to the subject a therapeutically effective amount of scopolamine.

2. The method of claim 1, wherein administration is further defined as placing a patch comprising a therapeutically effective amount of scopolamine on a surface of the subject, wherein delivery of scopolamine across the surface of the subject results in prevention or improvement of menopausal symptoms.

3. The method of claim 1, wherein delivery is further defined as transdermal delivery, transmucosal delivery, or transvaginal delivery.

4. The method of claim 1, wherein the subject is a mammal.

5. The method of claim 4, wherein the mammal is a human.

6. The method of claim 1, wherein the subject is further defined as a male or a female with hot flashes, night sweats, and/or insomnia due to hormonal changes.

7. The method of claim 6, wherein the subject is a female with one or more menopausal symptoms.

8. The method of claim 1, wherein the subject is further defined as a patient who has undergone chemotherapy, radiation therapy and/or hormonal therapy for the treatment of cancer.

9. The method of claim 8, wherein the cancer is breast cancer, endometrial cancer, or prostate cancer.

10. The method of claim 1, wherein the subject is a patient who has undergone surgery.

11. The method of claim 10, wherein the surgery is total abdominal hysterectomy with salpingo-oopherectomy, oopherectomy, or prostatectomy.

12. The method of claim 10, wherein the subject is a cancer patient.

13. The method of claim 12, wherein the cancer is breast cancer, endometrial cancer, or prostate cancer.

14. The method of claim 1, wherein the method further comprises identifying a subject in need of treatment or prevention of one or more menopausal symptoms.

15. The method of claim 14, wherein identifying a subject in need of treatment or prevention of hot flashes, night sweats, or insomnia comprises:
a) interviewing a subject to identify a subject affected by or at risk of developing hot flashes, night sweats, or insomnia; or
b) completion of a questionnaire by a subject to identify a subject affected by or at risk of developing hot flashes, night sweats, or insomnia.

16. The method of claim 1, wherein the subject is a person ineligible for estrogen therapy.

17. The method of claim 16, wherein the subject is ineligible for estrogen therapy because of a history of deep venous thrombosis, stroke, pulmonary embolism, myocardial infarction, endometrial carcinoma, or breast carcinoma.

18. The method of claim 2, wherein the patch is further defined as a transdermal delivery device.

19. The method of claim 18, wherein the transdermal delivery device comprises:
a. a backing layer;
b. a scopolamine reservoir; and
c. an adhesive layer.

20. The method of claim 18, wherein the transdermal delivery device is any of the transdermal delivery devices set forth in U.S. Pat. No. 5,714,162 or U.S. Pat. No. 6,537,571.

21. The method of claim 18, wherein the transdermal delivery device further comprises a membrane controlling the scopolamine flux from the patch to the surface of the subject.

22. The method of claim 19, wherein the scopolamine reservoir is further defined as a pressure sensitive scopolamine reservoir.

23. The method of claim 19, wherein the transdermal delivery device further comprises an additional skin adhesive layer.

24. The method of claim 23, wherein the transdermal delivery device further comprises a peel strip.

25. The method of claim 19, wherein the transdermal delivery device further comprises a protective film.

26. The method of claim 19, wherein the scopolamine reservoir comprises a composition to increase saturation solubility of the scopolamine therein.

27. The method of claim 19, wherein the scopolamine reservoir comprises a composition to decrease saturation solubility of the scopolamine therein.

28. The method of claim 19, wherein the scopolamine reservoir further comprises a composition to increase the solubility of the scopolamine therein.

29. The method of claim 2, wherein the patch comprises about 0.5 to about 5 mg scopolamine.

30. The method of claim 29, wherein the patch comprises about 1.0 to 3.0 mg scopolamine.

31. The method of claim 30, wherein the patch comprises about 1.5 mg scopolamine.

32. The method of claim 2, wherein the patch is applied at a frequency of about every 3 days.

33. The method of claim 1, further comprising administration of one or more secondary therapies for menopausal symptoms.

34. The method of claim 33, wherein the secondary form of therapy is hormonal therapy.

35. The method of claim 34, wherein the hormonal therapy comprises estrogen therapy, progesterone therapy, or a combination of estrogen and progesterone therapy.

36. The method of claim 2, wherein the patch further comprises one or more additional therapeutic agents for treatment or prevention of menopausal symptoms.

37. The method of claim 34, wherein the one or more additional therapeutic agents is selected from the group consisting of an estrogen, progesterone, a synthetic progestin, and bellergal.

38. The method of claim 37, wherein the estrogen is estrone, estradiol-17β, estriol, or a synthetic estrogen.

39. The method of claim 37, wherein the synthetic progestin is selected from the group consisting of medroxyprogesterone, hydroxyprogesterone, medrogestone and norethindrone.

40. The method of claim 1, further comprising administering one or more agents that counteract one or more side effects of scopolamine.

41. The method of claim 40, wherein the one or more side effects are selected from the group consisting of drowsiness, dry mouth, and dizziness.

42. The method of claim 40, wherein administration of scopolamine is further defined as placing a patch comprising a therapeutically effective amount of scopolamine on a surface of the subject, wherein delivery of scopolamine across the surface of the subject results in prevention or improvement of menopausal symptoms.

43. The method of claim 42, wherein the patch comprises at least one agent that counteracts one or more side effects of scopolamine.

44. The method of claim 2, further defined as a method of administering a composition comprising a therapeutically effective amount of scopolamine and one or more additional agents, wherein the composition comprises bellergal.
